SEQUENCE LISTING (38 sequences)

SEQ ID NO: 1 - DNA, 14 bp, Artificial Sequence
  Description of Artificial Sequence Synthetic oligonucleotide
ggatgaaaactgga

SEQ ID NO: 2 - DNA, 40 bp, Artificial Sequence
  Description of Artificial Sequence Synthetic oligonucleotide
ggcctccagttttcatccggtcccaaaagggtcagtgctg

SEQ ID NO: 3 - DNA, 37 bp, Artificial Sequence
  Description of Artificial Sequence Synthetic oligonucleotide
aaaagaatgtcagtgtgtcacgtatggacgaattcgc

SEQ ID NO: 4 - DNA, 41 bp, Artificial Sequence
  Description of Artificial Sequence Synthetic oligonucleotide
ggccgcgaattcgtcggtatgcactgtgtgtgacattcaaa

SEQ ID NO: 5 - DNA, 15 bp, Artificial Sequence
  Description of Artificial Sequence Synthetic oligonucleotide
gatcctcaaacgcgt

SEQ ID NO: 6 - DNA, 33 bp, Artificial Sequence
  Description of Artificial Sequence Synthetic oligonucleotide
ccagggttttcccagtcacgacacgcgtttgag

SEQ ID NO: 7 - DNA, 39 bp, Artificial Sequence
  Description of Artificial Sequence Synthetic oligonucleotide
ggcctccagaaaacatccacgggctctaggatagatcgc

SEQ ID NO: 8 - DNA, 14 bp, Artificial Sequence
  Description of Artificial Sequence Synthetic oligonucleotide
ggatgttttctgga

SEQ ID NO: 9 - DNA, 10 bp, Artificial Sequence
  Description of Artificial Sequence Synthetic oligonucleotide
ctcaaaccgt

SEQ ID NO: 10 - DNA, 34 bp, Artificial Sequence
  Description of Artificial Sequence Synthetic oligonucleotide
ccagggttttcccagtcacgacacggtttgagnn

SEQ ID NO: 11 - DNA, 32 bp, Artificial Sequence
  Description of Artificial Sequence Synthetic oligonucleotide
ccagggttttcccagtcacgacacggtttgag

SEQ ID NO: 12 - DNA, 22 bp, Artificial Sequence
  Description of Artificial Sequence Synthetic oligonucleotide
cagcactgacccttttgggacc

SEQ ID NO: 13 - DNA, 19 bp, Artificial Sequence
  Description of Artificial Sequence Synthetic oligonucleotide
tgctggccacaggccctgc

SEQ ID NO: 14 - DNA, 22 bp, Artificial Sequence
  Description of Artificial Sequence Synthetic oligonucleotide
tgcatgtgccagtgttgataaa

SEQ ID NO: 15 - DNA, 19 bp, Artificial Sequence
  Description of Artificial Sequence Synthetic oligonucleotide
gtgttgtgagccctgggaa

SEQ ID NO: 16 - DNA, 25 bp, Artificial Sequence
  Description of Artificial Sequence Synthetic oligonucleotide
agcctgagctgggcagacagtttcc

SEQ ID NO: 17 - DNA, 18 bp, Artificial Sequence
  Description of Artificial Sequence Synthetic oligonucleotide
cagccccacggtcacttc

SEQ ID NO: 18 - DNA, 19 bp, Artificial Sequence
  Description of Artificial Sequence Synthetic oligonucleotide
gccaaaacagacccagcct

SEQ ID NO: 19 - DNA, 18 bp, Artificial Sequence
  Description of Artificial Sequence Synthetic oligonucleotide
ccccagaaacgcgcgggc

SEQ ID NO: 20 - DNA, 20 bp, Artificial Sequence
  Description of Artificial Sequence Synthetic oligonucleotide
cttgccatctgcaattccct

SEQ ID NO: 21 - DNA, 20 bp, Artificial Sequence
  Description of Artificial Sequence Synthetic oligonucleotide
ctccatgaggctgtgggaag

SEQ ID NO: 22 - DNA, 16 bp, Artificial Sequence
  Description of Artificial Sequence Synthetic oligonucleotide
gcggctggctctgcgc

SEQ ID NO: 23 - DNA, 20 bp, Artificial Sequence
  Description of Artificial Sequence Synthetic oligonucleotide
atgaggctctttcccatgcc

SEQ ID NO: 24 - DNA, 19 bp, Artificial Sequence
  Description of Artificial Sequence Synthetic oligonucleotide
gccggattcaggatgcttt

SEQ ID NO: 25 - DNA, 22 bp, Artificial Sequence
  Description of Artificial Sequence Synthetic oligonucleotide
ctggcggagagactgggagcga

SEQ ID NO: 26 - DNA, 19 bp, Artificial Sequence
  Description of Artificial Sequence Synthetic oligonucleotide
cagagtgcgtgtgccgact

SEQ ID NO: 27 - DNA, 19 bp, Artificial Sequence
  Description of Artificial Sequence Synthetic oligonucleotide
acaacttctctgcgggcgt

SEQ ID NO: 28 - DNA, 25 bp, Artificial Sequence
  Description of Artificial Sequence Synthetic oligonucleotide
atgccccccccatgccatcctgcgt

SEQ ID NO: 29 - DNA, 25 bp, Artificial Sequence
  Description of Artificial Sequence Synthetic oligonucleotide
tcacccacactgtgcccatctacga

SEQ ID NO: 30 - DNA, 25 bp, Artificial Sequence
  Description of Artificial Sequence Synthetic oligonucleotide
cagcggaaccgctcattgccaatgg

SEQ ID NO: 31 - DNA, 12 bp, Artificial Sequence
  Description of Artificial Sequence Synthetic oligonucleotide
ggccgcnnnnnn

SEQ ID NO: 32 - DNA, 38 bp, Artificial Sequence
  Description of Artificial Sequence Synthetic oligonucleotide
ggatgaaaactggaggccgcnnnnnnnnnnnnnnnnnn

SEQ ID NO: 33 - DNA, 60 bp, Artificial Sequence
  Description of Artificial Sequence Synthetic oligonucleotide
nnnnnnnnnnnnnnnnnngcggcctccagttttcatccggtcccaaaagggtcagtgctg

SEQ ID NO: 34 - DNA, 31 bp, Artificial Sequence
  Description of Artificial Sequence Synthetic oligonucleotide
ggatgaaaactggaggccgcnnnnnnnnnnn

SEQ ID NO: 35 - DNA, 51 bp, Artificial Sequence
  Description of Artificial Sequence Synthetic oligonucleotide
nnnnnnnnngcggcctccagttttcatccggtcccaaaagggtcagtgctg

SEQ ID NO: 36 - DNA, 42 bp, Artificial Sequence
  Description of Artificial Sequence Synthetic oligonucleotide
ggatgaaaactggaggccgcnnnnnnnnnnnnctcaaaccgt

SEQ ID NO: 37 - DNA, 86 bp, Artificial Sequence
  Description of Artificial Sequence Synthetic oligonucleotide
ccagggttttcccagtcacgacacggtttgagnnnnnnnnnnnngcggcctccagttttcatccggtcccaaaagggtcagtgctg

SEQ ID NO: 38 - DNA, 86 bp, Artificial Sequence
  Description of Artificial Sequence Synthetic oligonucleotide
cagcactgacccttttgggaccggatgaaaactggaggccgcnnnnnnnnnnnnctcaaaccgtgtcgtgactgggaaaaccctgg

(19) United States
(12) Patent Application Publication
(10) Pub. No.: US 2005/0064406 A1
(43) Pub. Date: Mar. 24, 2005

(54) Methods for high throughput genome analysis using restriction site tagged microarrays

(76) Inventors: Eugene Zabarovsky, Upplands (SE); Ingemar Ernberg, Enebyberg (SE); Jingfeng Li, Solna (SE); Alexei Protopopov, Brookline, MA (US); Claes Wahlestedt, Stockholm (SE); Vladimir Kashuba, Spanga (SE); Veronika Zabarovska, Upplands Vasby (SE)

Correspondence Address: YOUNG & THOMPSON, 745 SOUTH 23RD STREET, 2ND FLOOR, ARLINGTON, VA, 22202, US

(21) Appl. No.: 10/475,352

(22) PCT Filed: Apr. 22, 2002
(86) PCT No.: PCT/SE02/00788

Related U.S. Application Data
(60) Provisional application No. 60/284,925, filed on Apr. 20, 2001.

(51) Int. Cl. C12Q 1/68; C12P 19/34

(52) U.S. Cl. 435/6; 435/91.2

(57) ABSTRACT

A method for high-throughput analysis of genomic material originating from complex biological systems, including complex microbial systems and a method of detecting changes in a genomic material using restriction site tagged (RST) microarrays and sequence passporting technique (in particular microarrays containing NotI-clones). Using the present invention method, methylation or silencing of specific alleles, homozygous and hemizygous deletions, epigenetic factors, genetic predisposition, etc, information which is particularly useful in diagnosis and treatment of cancer diseases, can be detected. The RST microarrays and passporting can also be used for qualitative and quantitative analysis of complex microbial systems.

### FIELD OF THE INVENTION

The present invention pertains to a method of detecting changes in a genomic material using restriction site tagged (RST) microarrays and passporting technique, which can be used for detecting methylation or silencing of specific alleles, homozygous, hemizygous deletions, epigenetic factors, genetic predisposition, etc, information which is particularly useful in diagnosis and treatment of cancer diseases. The RST microarrays and passporting according to the present invention can also be used for qualitative and quantitative analysis of complex microbial systems.

### BACKGROUND OF THE INVENTION

Genomic subtractive methods in principle are very useful for identification of disease genes including tumour suppressor genes. However, among many suggested techniques only a modified variant of genomic subtraction called Representational Difference Analysis (RDA, Lisitsyn et al., 1993) and RFLP subtraction (Restriction Fragment Length Polymorphism)(Rosenberg et al., 1994) have been reproducibly succesful in cloning deleted sequences. Three main drawbacks limited wide use of these related methods: both are very complicated and laborious, they are very sensitive to minor impurities and experiments result in cloning only a few deleted sequences. It is important to note that these methods only work well with enzymes not being associated with CpG islands. Methylation-sensitive-representational analysis (MS-RDA, Ushijima et al., 1997) has more specific aims, i.e. they work with CpG Islands, but still is not avoided limitations of the original RDA. Moreover, differentially cloned products usually do not have any connections with genes. Deletions of non-functional regions occur frequently in the human genome and cloning of such segments will not yield valuable information (Lisitsyn et al., 1995). RDA is also unable to detect differences due to point mutations, small deletions or insertions, unless they affect a particular restriction enzyme recognition site. Another source of artefacts is the PCR amplification after the first hybridization step and before the nuclease treatment. The presence of excess driver DNA can result in a reduced efficiency of the amplification tester:tester duplexes due to the opportunity for the residual driver:driver and driver:tester duplexes to act as competitors. As RDA is based mainly on specific PCR amplification of desired products and use many cycles (95-110), it suffers from a “plateau effect” that is characterised by a decline in the exponential rate of accumulation of amplification products (Innins and Gelfand, 1990). However, the major problem results from the inefficiency of the multiple restriction digestion and ligation reactions that are used in this method and leads to the generation of false positives.

The presence of genetic alterations in tumours is now widely accepted, and explains the irreversible nature of tumours. However, observations on tissue differentiation indicated that it shares something in common with carcinogenesis, i.e. “epigenetic” changes. Now, DNA methylation in CpG sites is known to be precisely regulated in tissue differentiation, and is supposed to be playing a key role in the control of gene expression in mammalian cells. The enzyme involved in this process is DNA methyltransferase, which catalyzes the transfer of a methyl group from S-adenosyl-methionine to cytosine residues to form 5-methylcytosine, a modified base that is found mostly at CpG sites in the genome. The presence of methylated CpG islands in the promoter region of genes can suppress their expression. This process may be due to the presence of 5-methylcytosine that apparently interferes with the binding of transcription factors or other DNA-binding proteins to block transcription. DNA methylation is connected to histone deacetylation and chromatin structure, and regulatory enzymes of DNA methylation are being cloned.

In different types of tumours, aberrant or accidental methylation of CpG islands in the promoter region has been observed for many cancer-related genes resulting in the silencing of their expression. The genes involved include tumour suppressor genes, genes that suppress metastasis and angiogenesis, and genes that repair DNA, suggesting that epigenetics plays an important role in tumourigenesis. The potent and specific inhibitor of DNA methylation, 5-aza-2-deoxycytidine (5-AZA-CdR) has been demonstrated to reactivate the expression of most of these malignant suppressor genes in human tumour cell lines. These genes may be interesting targets for chemotherapy with inhibitors of DNA methylation in patients with cancer, and may help to clarify the importance of this epigenetic mechanism in tumourigenesis. Spontaneous regression of malignant tumours used to enchant researchers, but it has now been observed that genes inactivated by hypermethylation are frequently involved in tumours that relatively often undergo spontaneous regression. Carcinogenic mechanisms of some carcinogens seem to involve modifications of an epigenetic switch, and some dietary factors also have the possibility to modify the switches.

Review articles in the literature make it clear that methylation is a basic, vital feature/mechanism in mammalian cells. It is involved in hereditary and somatic cancers, hereditary and somatic diseases, apoptosis, replication, recombination, temperature control, immune response, mutation rate (i.e. in p53). Through methylation food can induce cancer, etc., it is believed that it can be used for diagnostic, prognostic, prediction and even for direct treatment of cancer. Inactivation of DNA methyltransferase is lethal for mice. Based on the growing understanding of the roles of DNA methylation, several new methodologies have been developed to make a genome-wide search for changes in DNA methylation.

There are four main genome-wide screening methods (see Sugimura T, Ushijima T, 2000) for testing methylation in human genome: restriction landmark genomic scanning (RLGS, Costello et al., 2000), methylation-sensitive-representational difference analysis (MS-RDA), methylation-specific AP-PCR (MS-AP-PCR) and methyl-CpG binding domain column/segregation of partly melted molecules (MBD/SPM). Although each of them has their own advantages, none of them is suited for large-scale screening since all four are rather inefficient and complicated; they can be used only for testing a few samples. For example, after analysis of 1000 clones isolated using MBD/SPM, nine DNA fragments were identified as CpG islands and only one was specifically methylated in tumour DNA.

Recently developed microarrays of immobilized DNA open new possibilities in molecular biology. These DNA arrays, containing either cDNA or genomic DNA, are fabricated by high speed robotics on glass substrates. Probes that are labeled by different colors are hybridized. In one such hybridization thousands of genes or genomic DNA fragments can be analyzed allowing massive parallel gene expression and gene discovery studies. In pilot experiments microarrays with immobilized P1 and BAC clones DNA demonstrated that they could be used for high resolution analysis of DNA copy number variation using CGH (comparative genome hybridization). It has been suggested that this approach can work if inserts of human DNA in the cloning vectors are larger than 50 kb. In the future, when microarrays with P1 and BAC clones covering the whole human genome will be created, this approach will most likely replace coventional CGH. Clearly, construction of such microarrays with mapped P1 and BAC clones is very expensive, laborious and time consuming. Construction of such microarrays cannot be achieved in a single research laboratory. If small-insert NotI liking clones could full the same function this will open the way to construct such microarrays for CGH analysis for a single research group and for many organisms. PACs and BACs covering the whole human genome are not available yet.

Pollack et al., 1999 suggested to use cDNA microarrays for genomic DNA copy number changes but small size of cDNA clones and high ratio of background hybridization compared to real signal makes this suggestion problematic.

In the fall 2000 Affymetrix launched the selling of GeneChipHuSNP Mapping Assay. These microarrays contain 1.494 SNP loci. In the promotion papers it was shown that this microarrays can be used for the detection of loss of heterozygosity (LOH). However 13% of SNPs failed in the majority of samples whereas only 354 SNPs were informative in one particular experiment.

Lucito et al. (2000) used for the detecting copy number fluctuations in tumour cells modification of RDA technology. In this method BglII representations were used in conjunction with DNA microarrays. As there are many small BglII clones in the human genome (150.000) it will be not easy and cheap to make comprehensive microarrays with unique clones covering the whole human genome.

Presently, there are some methods available to analyze complex microbial mixtures, e.g. by enzyme analysis (Katouli et al., 1994) which requires growth of colonies outside the body, or analysis of the composition fatty acids in stools which gives crude indications of the composition of the normal flora (refs.), however all them have obvious limitations.

The application of culture-independent techniques based on molecular biology methods that can overcome some shortcomings of conventional cultivation methods. In recent years the approaches based on PCR amplification of 16S rRNA genes have been most popular. One modification of the approach utilized fingerprinting of all the species in the gut using, for instance, denaturing gradient gel electrophoresis (DGGE) with PCR amplified fragments of 16S rRNA genes. In another application, PCR amplified fragments of 16S rRNA genes were directly cloned and sequenced. These studies yielded important information however intrinsic disadvantage of the approach limits its application. The problem is that 16S rRNA genes are highly conserved and therefore the same sequenced fragment can belong to different species. It is also important to keep in mind that in fingerprinting experiments similar fragments can represent different species, and different fragments can represent the same species.

### SUMMARY OF THE INVENTION

In view of the drawbacks associated with the prior art methods for analysis of genomic material originating from complex biological systems, there is a need for uncomplicated, quick and reliable genome analysis methods.

Therefore, the object of the present invention is to provide novel and unique techniques for analysis of genomic material originating from complex biological systems, including complex microbial systems. The main objects of the present invention are the following:

One object of the present invention is to prepare and to use NotI-clone (in general PCR fragments, oligonucleotides, etc.) microarrays for studying methylation and/or copy number changes in eukaryotic genomes for diagnosis, prognosis, identification of cancer causing genes. NotI microarrays are the only existing microarrays giving the opportunity to detect copy number changes and methylation simultaneously. This includes comparison of normal and malignant cells at genomic and/or RNA level; comparison of primary tumours and metastases; analysis of families suffering from hereditary diseases including cancers; and diagnostics and disease prediction.

Capability to establish differences between normal and tumour cells is instrumental for cloning cancer causing genes and for early diagnosis and prevention of cancer. It is also very important for differentiation, development and evolution studies.

Another object of the present invention is to provide techniques allowing qualititative and quantitative analysis of complex microbial systems, such as the normal flora of the gut.

A further object of the present invention is to prepare NotI sequencing passports (“NotI passport”) (collection of NotI tags: short sequences surrounding genomic NotI sites) and to use them to study the same problems as were mentioned above for NotI microarrays.

Wide screening of genomic material using RST encounter many problems, e.g. the size of the human genome/microbial mture and the number of repeat sequences. We have solved these problems by developing a new method for labeling genomic DNA, where only sequences surrounding NotI (or any other restriction) sites are labeled (tagged), herein called NotI Representation (NR).

In the present invention, Restriction Site Tags (RSTs) are generated from thousands of microorganisms or human genomes and used for the generation of NotI RST microarrays passports which describe uniquely not only individual human cell/organism or bacterial strains but most or all the members of a microbial flora of e.g. in the gut.

With the NotI or RST genome scanning method according to the present invention, large scale scanning of microbial genomes on a quantitative and qualitative basis is possible.

From the results of our experiments, we have shown that it is possible to create a large database containing NotI microarrays passports, i.e. NotI microarray images. Many samples of colon flora have been compared to determine their exact composition.

The present invention procedure is universal, i.e. we can use any other enzyme for creating “RST microarray passports”. Moreover, any biochemical or chemical approach cutting DNA (RNA) in a specific position scarcely distributed along DNA (RNA) can be used. For example, it can be enzyme like cre-recombinase or chemically modified oligonucleotide forming triplex DNA and initiating DNA break. The polymorphism of NotI representations can be increased by using several enzymes in addition to BamHI, e.g. BclI, BglII, HindIII etc. In pilot experiments we have produced NotI microarrays from gram-positive and gram-negative bacteria and have shown that even very similar E. coli strains can be easily discriminated using this technique. Using the above mentioned technique we can identify important pathogenic bacteria in the human organism.

These ‘NotI microarrays passports’ can be produced for individuals, normal/tumour pairs, different cell NotI Representation (NR). A pilot experiment using NR probes demonstrated the power of the method, and we successfully detected Chr.3 NotI clones deleted in ACC-LC5 and MCH939.2 cell lines.

Such NotI RST microarrays can be prepared for any human or any groups of humans, who for example suffer from the same specific disease, in order to detect a certain disease which cannot be detected by other means. NotI RST microarrays can also be prepared for any mammal (like cattles or dogs) or microbial organism.

NotI arrays will speed up cancer research very significantly and can replace CGH, LOH and many cytogenetic studies.

The NotI scanning approach will find mainly deleted, amplified, or methylated genes but it will also identify polymorphic and mutated NotI sites. Comparing these NotI passports can give a clue to understanding many diseases and other fundamental biological processes.

Using the present invention method of producing RST microarrays, restriction enzyme tagged (RST) microarrays for any enzyme can be created. The microarrays according to the present invention represent a novel type of microarrays, which is completely different from the existing ones (oligonucleotides, cDNA, genomic BAC/PAC clones).

To be able to establish differences between individual compositions of the normal gut flora will be instrumental for future analysis of how the normal flora composition is influenced by diet, special foods, geographical location, colon, ovarian, etc. cancers and other diseases. It has particularly wide applications for cancer research.

The present invention method will probably have strong impact both on basic science and on human and animal health, agriculture, medicine, pharmacology, etc.

We propose to use our NotI clones as a complement to microarrays based on P1 and BAC clones covering the whole human genome. Microarrays based on small-insert NotI linking clones have been developed, and can have a similar function. Approximately 10.000-20.000 NotI clones, covering the whole human genome and containing 10%-20% of all genes (40%-50% of them are not present in ESTs microarrays) are already available.

In order to achieve what is described above, the present invention comprises the following embodiments:

In one embodiment of the present invention provides a method for preparing nucleic acid or and/or modified nucleic acid reference material bound to a solid phase, comprising the steps of
digesting nucleic acid and/or modified nucleic acid reference material using biochemical and/or chemical approaches, to obtain sequence fragments surrounding a specific recognition site,
selecting said nucleic acid and/or modified nucleic acid sequence fragments associated with a specific recognition site.

Said reference material is digested by a first restriction enzyme and/or one or more second restriction enzymes, e.g. endonucleases, such as cre-recombinase,

In one embodiment of the present invention the recognition sites of the first endonuclease is scarcely distributed along said genomic material and is located adjacent to gene sequences, and the recognition sites of said one or more second restriction endonucleases are more frequently occurring along said genomic material than the sites of the first endonuclease.

In another embodiment of the present invention the digestion by the first and second restriction endonucleases are performed simultaneously, and different linkers are ligated to the ends resulting from cutting by the first and second restriction endonucleases, respectively, which linkers are designed such that when primers are added in order to make PCR reactions, only the fragments containing ends resulting from cutting by the first restriction endonuclease will be amplified.

In still another embodiment of the present invention the reference material is first digested by the one or more second restriction endonucleases, the ends of the thus obtained fragments are self-ligated into the form of circular nucleic acid and/or modified nucleic acid molecules, and any linear fragments remaining after self-ligation are inactivated before digestion with the first restriction endonuclease, whereby the linear fragments resulting from the digestion by the first endonuclease are subjected to PCR amplification.

In these embodiments the first restriction endonuclease is NotI, or any other restriction endonuclease, the restriction sites of which occurs in proximity to CpG islands in the genomic material.

The first restriction endonuclease can also be NotI, PmeI or SbfI, or a combination of two or more of said endonucleases, and the second endonuclease can be BamHI, BclI, BglII or Sau3A, or a combination of two or more of said endonucleases.

Said nucleic acid and/or modified nucleic acid reference material can be selected from RNA, DNA, peptides or modified oligonucleotides, or a combination of two or more of said materials.

In the present invention nucleic acid and/or modified nucleic acid is bound to a solid glass support in the form of a microarray. However, the present invention is not limited to using glass microarrays. Solid phases such as filters, e.g. nylon filters, coded beads, cellulose, such as nitrocellulose, or other solid supports can also be used to bind nucleic acid and/or modified nucleic acid. In general DNA, oligonucleotides, etc. bound to a solid phase can be used.

The genomic material that can be used according to the present invention can be derived from one or more humans, from different locations in the body/bodies and at the same or different points in time. Said genomic material can be derived from bacteria from the gut, skin or other parts of the human body. However, it can also be derived from any organism, bacteria, animal, or plant, or product produced therefrom, or from any substance wherein genomic material can be contained, especially air and water.

The present invention also pertains to the fragments that can be obtained using the present invention, and the nucleic acid or and/or modified nucleic acid microarrays containing these fragments.

The present invention further pertains to representations of the genome, or of a part thereof, of an organism, comprising multiple copies of the nucleic acid and/or modified nucleic acid fragments, or a selection thereof, obtained by means of the present invention method.

These representations, in liquid form, are hybridized to the nucleic acid and/or modified nucleic acid fragments present in the form of said solid phases.

Said representations can be used for discriminating between different genomes, detecting methylations, deletions, mutations and other changes within genomic material obtained from the same individual at different points of time, or in the genomic material obtained from one individual as compared to a standard representation obtained from at least one other individual, or a combination thereof.

In addition to the above-mentioned applications, these representations can be used for:
studying methylation and copy number changes in eukaryotic genomes for diagnosis, prognosis, identification of cancer causing genes, etc,
genotyping different microorganisms (viruses, prokaryotic, eukaryotic),
studying biocomplexity and diversity of complex biological systems, i.e. human gut, bacterial flora in water, food, air resources,
identifying pathogenic organisms in different sources including complex biological mixtures,
producing passports (images of microarrays hybridizations, databases containing tag sequences) for different purposes: to describe organisms at different conditions, i.e. different ages, disease/healthy, infected/uninfected etc,
identifying new organisms, e.g. bacterial species,
producing microarrays (DNA- and oligo-based) to study all above described features,
verification and maintenance of large biological collection/banks, i.e. verifying cell lines and individual organisms for higher organisms and confirming the purity of the particular strain for microbial species,
producing kits for labeling and hybridization with microarrays,
producing kits for making sequence tagging (passporting), and
producing oligo microarrays to analyze sequence tags,

Finally, the present invention also pertains to a NotI CODE genomic subtraction method based on the use of the above described fragments.

### BRIEF DESCRIPTION OF THE DRAWINGS

FIG. 1. General scheme for the NotI-CODE subtractive procedure.

FIG. 2. Southern hybridization of NotI clones showed different hybridization. Clone names are shown at the bottom. N—normal DNA, L—DNA isolated from lung cancer cell line ACC-LC5.

FIG. 3. General principle of using NR for NotI microarrays.

FIG. 4. NotI microarrays profiling of deletions/methylation in microcell hybrid MCH 939.2 (A), cell line ACC-LC5 (B), and primary RCC tumors #196 (C) and #301 (D). Representative images of microarrays (1) are ordered according to physical map of chromosome 3. One-dimensional clustering (2) is based on average normalized red/green ratios of fluorescent data (red, R>3; green, R<0.3). For (A) and (B) normal and tested DNA were hybridized together. NR for MCH903.1 (the whole chromosome) was labeled red and NR for MCH939.2 (3p.14-p22 deletion) was labeled green. Similarly, NR for normal lymphocyte DNA was red and small cell lung cancer line ACC-LC5 was labeled green. The red clusters demonstrate a significant overrepresentation of complete chromosome 3 or normal DNA. The green clusters—under representation of normal DNA. For (C) and (D) one step of NotI-CODE subtraction procedure was performed and single color hybridization was done. The green clusters demonstrate the significant overrepresentation of normal DNA. Grey color marks controls.

FIG. 5. General scheme of the experiment. (microbial flora)

FIG. 6. Flow chart diagram explaining generation of 85 bp oligonucleotide containing information about 19 bp NotI-tag

### DETAILED DESCRIPTION OF THE INVENTION

In the literature it has been suggested and demonstrated that NotI sites are practically exclusively located in CpG islands and are closely associated with functional genes. Thus NotI sites are very useful markers not only for physical but also for genetic mapping.

The present inventors have created high-density grids that contain 50.000 of NotI clones originating from 6 representative NotI linking libraries and generated more than 22.000 unique NotI sequences (with stringent criteria 16.000) containing 17 Mb information. Analysis of these sequences demonstrated that even short sequences surrounding NotI sites is a source of important information allowing efficient isolation of new genes and the study of carcinogenesis.

We have a developed new approach for constructing NotI lining libraries (Zabarovsky et al., 1990) that give possibility to generate representative NotI linking libraries both in lambda phage and in plasmid form (Zabarovsky et al., 1994a). Since the procedure is quite easy and reproducible, it is possible to construct libraries from many sources.

Using the present invention NotI (RST) microarrays, based on the short sequences surrounding NotI sites or in general on restriction site tagged sequences (RSTS), complex biological systems, including complex microbial mixtures, can be qualitatively and quantitatively analysed.

In the present invention study NotI microarrays for human chr.3 (150 clones) were established and employed to compare chr 3 renal, lung, breast and nasopharyngeal cancers.

NotI Microarrays for Genome Wide Scanning

Recently we have sequenced 25.000 NotI clones and identified among them 16.000 unique clones. These clones that cover the whole human genome and contain 10%-20% of all genes (40%-50% of them are not present in ESTs microarrays) are already available.

The NotI microarrays can be used for testing tumour genomic DNA in genome wide NotI scanning (e.g. for deletion/amplification studies). Such arrays will speed up cancer research very significantly and can replace LOH (loss of heterozygosity), CGH (comparative genome hybridization), and other cytogenetic studies.

The fundamental problems for genome wide screening using NotI clones are:
(i) the size and complexity of the human genome;
(ii) the number of repeat sequences; and
(iii) the comparatively small size of the inserts in NotI clones (on average 6-8 kb).

To solve this problem, the special primers were designed and special procedure was developed to amplify only regions surrounding NotI sites, so called NotI representation (NR). Other DNA fragments were not amplified. We suggested to use NotI microarrays for genome screening in combination with this new method for labeling genomic DNA where only sequences surrounding NotI sites are labeled.

NotI microarrays images can be generated for particular cells, tumours, and individuals. By comparing images from normal and tumour cells, the differences between them will be defined. Using this information, NotI linking clones will be identified that differ between two (or more) DNAs. These clones can be used for further analysis and for isolating complete genes. Polymorphism in NotI sites is very frequent and according to the literature 43.5% of NotI sites are differently methylated or polymorphic.

Analysis of our database of 16.000 unique NotI sequences (two sequences can belong to the same NotI clone) showed that practically all of them are connected with genes and located at the 5′ end of the genes. Comparison with completely sequenced chr. 21 and 22 revealed interesting observations. Chr. 21 contains 122 NotI sites (methylated and unmethylated) and Ichikawa et al., 1993 have cloned 40 NotI sites to construct the complete NotI restriction map with 43 NotI fragments. From these 40 clones our database contained 38 (95%) and additional 13 NotI clones (11%). Therefore using random sequencing we could isolate 27.5% more NotI clones than in the study of Ichikawa et al., 1993 where they focused their efforts in cloning NotI clones only from chr. 21. Altogether, from 390 possible NotI sites in chr. 21 and 22 our database contain 163 (42%) clones. Moreover, 18 clones that were identified in our work (5%) were not present in public sequences. These clones contained polymorphic NotI sites. Thus, from our data we can conclude that unmethylated (our database contain only unmethylated NotI sites) NotI sites represent appr. 42% and polymorphic —5% of all possible NotI sites. Our estimation is that human genome contains 15.000-20.000 NotI sites and 6.000-9.000 of them are unmethylated in a particular cell. Thus screening with NotI microarrays will be equivalent to screening using 6.000-9.000 gene associated single nucleotide polymorphisms (SNP).

Comparing the prior art genomic chips with the present invention NotI microarrays it is easy to see that NotI microarrays give additional information to the deletion mapping: they can be used for gene expression profiling and methylation studies (see Table 1).

For preparing the probe for SNP chip 3.000 PCR primers and 24 separate reactions are needed and probe for NotI microarrays is prepared using 1-2 primers in one reaction tube. Using the same NotI clones we are able to simultaneously obtain information about:
(i) deletions/amplifications;
(ii) methylation;
(iii) gene expression profiles.

All these features of NotI microarrays are extremely important for large scale experiments.

The pattern of hybridization of NR to the NotI microarrays represent a microarray passport for the DNA used for preparing NR.

We will now summarize the differences between CpG islands microarrays (below abbreviated to CGI, see Yan et al., Cancer Res. (2001) 61: 8375-8380), which we presently find is the closest prior art, and the present invention RST microarrays (below abbreviated to RST, see Table 2).

In the present invention sequences surrounding the same restriction site are cloned, whereas in CGI sequences originate from sequences between two restriction sites.

In principle, using the present invention technique, any restriction enzyme can be used for RST, but only limited number for CGI.

CGI can detect methylation, but not (in general) deletions (hemi- or homozygous) or amplifications of unmethylated sequences. RST can detect both copy number changes and methylation. CGI can detect deletion of the allele if it is methylated in normal genomic material and if it is deleted (unmethylated) in tumour material, this process is however inefficient as the vast majority of the important genes are unmethylated in normal genomic material, and the majority of methylated genes in normal genomic material are various kinds of repetitive elements, e.g. LINE, Long Interspersed Element (or sequence or repeat).

In CGI the total human DNA is labeled, in RST only 0.1-0.5%, and this DNA contains 10-fold less repeats than the total human DNA.

Many clones in CGI contain repeats and ribosomal DNA, whereas the RST only comprise genes containing unique human sequences. This very important difference is the result of completely different techniques of constructing microarrays (they use methyl-CG binding column, which is not used in the present invention).

For RST microarrays short OLIGOS (oligonucleotides 20-100 bp) can be used, which is not possible for CGI.

Incomplete digestion do not create problems for RST, but produce artificial signals in CGI.

Using RST hybridization is obtained when the site is not methylated, whereas in CGI hybridization only occurs if it is methylated.

CGI microarrays can only be used to study methylation in high vertebrates. This can also be done with RST, which in addition to that, also can be used for genotyping (passporting) any organism. It means that RST microarrays can be used to genotype bacteria and viruses for example, but not CGI.

Our RST application contains complementary aspects, i.e. the generation of NotI (RST) tags (passports) by sequencing. Sequencing can be done using different techniques including sequencing by hybridization to microarrays. No such complementary approach is possible with CGI.

NotI-CODE (or RST-CODE in general) can be used together with RST microarrays to remove in one step contaminating sequences. No such technique can be applied for CGI. Existing subtractive procedures like RDA cannot be employed, since they are not efficient enough to deal with the high complexity of total human genomic DNA.

Using RST microarrays it is possible to discriminate between deleted/amplified and methylated sequences. To achieve this aim NR should be produced using DNA that is unmethylated (it can be done by different approaches: limited PCR amplification after first digestion with restriction enzyme(s), enzymatic demethylation, etc.).

NotI Passporting

We originally planned to use SAGE technique for this purpose. Serial analysis of gene expression (SAGE) allows for both a representative and comprehensive differential gene expression profile (Velculescu et al., 1995). The idea of the approach is that for each of the mRNA molecule a short 9-bp sequence tag is produced (including recognition site for the tagging enzyme it is 13 bp). Then these tags are ligated into concatemers and cloned. One sequencing reaction produces information for tens of RNA molecules. Thus by sequencing a few thousands clones one can e.g. evaluate all of the estimated 10.000 to 50.000 expressed genes in a given cell population. We have tried the SAGE technique for producing NotI tags but this was unsuccessful. Complexity of genomic DNA in microbial mixtures is at least 100 times more complex than the complexity of mRNA in eukaryotic cells. All RNA molecules must be tagged in SAGE but in our case, approximately one out of 250 molecules should be tagged. We propose to produce one tag for each 100-1.000 kb, but in SAGE one tag is produced for 256 bp. At the same time, a 13 bp tag is not enough for unambiguous identification of sequences in genomic DNA. That is why we have developed a new procedure called Not passporting.

In this work we used the following modification. Genomic DNA was digested with NotI and ligated to the linker with NotI sticky ends. This linker contained BpmI recognition sites. This restriction nuclease cut 16/14 bp outside of the recognition site. Ligation mixture was digested with this enzyme to generate 11/9 nucleotide tags adjacent to the NotI site. This DNA sample was ligated to ZNBpm linker and PCR amplified with antiuniver and Z1univer primers to generate 85 bp duplex. The final PCR amplified molecule contains 17 bp sequence tag which is missing 2 bp from the original NotI site and therefore the whole NotI tag contains 19 bp. NotI passports were experimentally produced for E. coli K12, E. cloaceae R4 and K. pneumoniae B4958. Experiments with samples obtained from mice demonstrated that the quality of DNA isolated from intestine of feces was sufficient to obtain NotI tags. The NotI passports uniquely identified these species and among 96 tags none was common for these 3 bacterial species. Of course, ditags or concatemers also can be created from these 85 bp products. We believe that new high-throughput technologies like MPSS will make sequencing of single tags more efficient approach than creation of concatemers. However, the design of the experiments can be different in different laboratories. As we mentioned above, this restriction site tagging procedure can be adapted to any recognition site for restriction nuclease. For comprehensive analysis of flora composition, use of several passports will be advantageous: different bacteria possess very different CG content. It means that with NotI passports bacteria having high CG content (NotI recognition site: GCGGCCGC) will predominantly be represented, but using for example SwaI passports (Swal: ATTTAAAT), bacterial genomes with high AT content will be analyzed more carefully. Use of 2-3 different passports can significantly increase the sensitivity of the analysis and also be favourable for different applications, e.g. cancer risk, medication, diet, etc.

We tested the potentiality of the passporting approach and analyzed 25 bacterial species that were completely sequenced. The number of recognition sites for rare cutting restriction enzymes in these bacterial species are given in Table 3 below. It is easy to see that all 25 microbial species have different number of NotI recognition sites and therefore can be distinguished by NotI passporting. Moreover, from the Table 3 we can see that PmeI and SbfI restriction enzymes were even more informative.

Table 4 showed results of comparisons of different strains of E. coli and Helicobacter pylori for NotI, PmeI and SbfI enzymes. All of these strains were uniquely described by any of these enzymes and thus the inventive method can really discriminate between different species and strains, which was not possible with 16S rRNA genes sequencing.

All sequenced E. coli strains contained altogether 1 312 tags (including the tags to the left and to the right of the NotI recognition site) for these 3 enzymes, and among them only 139 were not unique. We can take into the account that two tags describe the same NotI site and therefore one tag can be the same but another can be different and therefore both tags still represent a unique NotI site. In such a case only 82 tags were not unique. These results demonstrate the power of the approach.

In our comparative experiments we did not use only bacterial genome sequences but the whole human genome sequences (including EST and EMBL entries). In such experiments, in the majority of the cases, NotI tags were unique even with the allowance of 1-2 sequence mismatches.

As mentioned above, the strongly advantageous feature of NotI passporting is the internal control. If a NotI site from a particular bacterial species contains for example NotI tag100 and NotI tag 101, then both tags should be obtained in approximately the same quantities. If only NotItag100 is present, then it most probably means that NotItag100 originates from another bacterial species.

The CODE procedure mentioned above can efficiently be applied to the NotI flanking sequences (Li et al., Proc. Natl. Acad. Sci. USA, (2002) in press). Thus, the power and sensitivity of the passporting procedure can be significantly increased by removing the most abundant species with the CODE technique (Li et al., 2001).

To be able to analyze complex microbial mixtures can be important for many applications. For instance, differences between individual composition of the normal flora will be instrumental for future analysis of how the normal flora composition is effected by diet, special foods, geographical location, colon diseases, autoimnunity, bacterial effects on colonic cancer risk, medication such as antibiotics and development of probiotics.

For this analysis we suggest to use generated restriction site tagged sequences. Hundreds of thousand tags can be produced in a short time, allowing careful analysis of thousands of bacterial species/strains (Velculesku et al., 1995). We have demonstrated that such NotI tags can be efficiently produced and that such tags have high specificity. The power of the method can be increased using the CODE subtractive procedure. We also provide a database for ‘NotI passports’ (as it was mentioned above it is more correct to speak about ‘RSTS passports’). Such database can be used together with a NotI (RST) microarrays database (Li et al., Proc. Natl. Acad. Sci. USA, (2002) in press) as these approaches are mutually complementary. This integrated database generates new knowledge as these two approaches are based on completely different biochemical techniques but aim to solve the same problem.

NotI—CODE Subtraction

Prior to the present invention, the inventors developed a new genomic subtraction procedure called CODE, Cloning Of Deleted Sequences (Li et al., Biotechniques, (2001), 31: 788-793) that does not suffer from some of the limitations of RDA and RFLP subtraction. The CODE is based on the modification of the COP procedure, (Li, J., Wang, F., Zabarovska, V., Wahlestedt, C., Zabarovsky, E. R., 2000, Cloning of polymorphisms (COP): enrichment of polymorphic sequences from complex genomes. Nucleic Acids Res.), which is a new procedure for cloning single nucleotide polymorphisms. Our major objectives were to develop a simple and reproducible procedure, and to improve subtractive enrichment, thereby avoiding excessive PCR kinetic enrichment steps that often generate small DNA products.

In the CODE procedure, a combination of digestion with restriction enzymes, treatment with uracil-DNA glycosylase (UDG) and mung bean nuclease, PCR amplification and purification with streptavidin magnetic beads, were used to isolate deleted sequences from the genomes of two human samples. The CODE has proved to be a rather simple, efficient and robust procedure.

In the present invention two questions had to be answered:
i) is it possible to use the CODE procedure for restriction enzymes containing CG in their recognition site and
(ii) is it possible to use NotI clones for genome wide screening for deleted, amplified and methylated NotI sites.

If the CODE procedure would work for the enzymes cutting in CpG islands, then it would be possible to clone not just deleted sequences (probably deleted by chance and without any meaning), but also genes that can be assumed as being candidate disease genes.

We suggest to use only regions surrounding NotI sites for subtraction. The novelty of this approach is that these regions are enriched and purified using circularisation. We have designed special primers and a procedure to obtain the NotI representations (NR). The other principles for this subtraction were the same as in the CODE procedure but genomic DNA was digested with BamHI+BglII and NotI and other linkers were used to allow PCR amplification of fragments containing only NotI. Other DNA fragments were not amplified. Only two cycles of subtraction were used here.

To validate this approach, we compared a lung tumour cell line ACC-LC5 that contained a 0.7 Mb homozygously deleted region in 3p21-p22, with normal lymphocyte control DNA. We did not know if this cell line contained homozygous deletions in other chromosomes. This normal DNA is not a completely appropriate control because it was isolated from another individual. We expected cloning of polymorphic sequences as well as deleted.

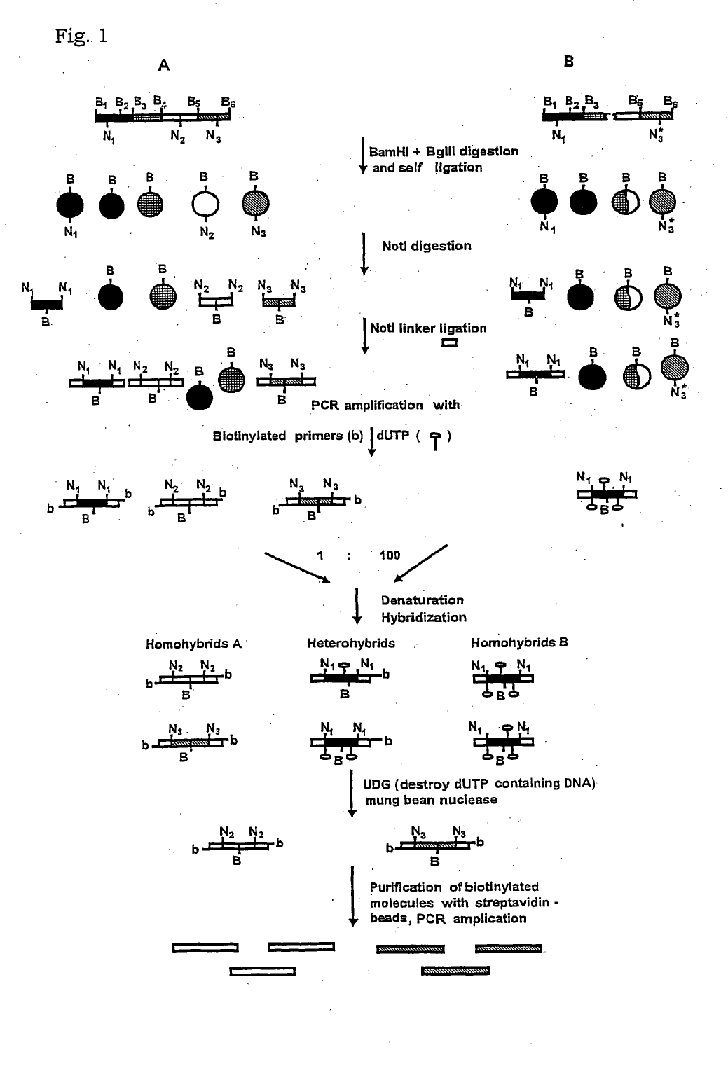

An overview of the subtractive procedure is shown in FIG. 1. Tester and driver DNA 15 was digested with BamHI+BglII and self-ligated at very low concentration of DNA to form circles. Intermolecular ligation does not create any problems because the vast majority (99.99%) of these ligated molecules will be not PCR amplified in the further steps. Even rare cases, such as when these two ligated molecules contain closely located NotI sites and will be able to be PCR amplified, are useful, since they serve to normalize the representativity of different NotI surrounding sequences. Then these circles were digested with NotI. The majority (approximately 99.9%) of the circles will not be opened and thus will be omitted from further reactions. This serves also to decrease background hybridization due to illegitimate ligation of NotI linker to the DNA fragments with BamHI or BglII sticky ends.

The driver DNA was amplified with dUTP and unmodified primers and tester DNA were amplified with biotinylated primers in the presence of normal dNTPs. The products of DNA amplification (on average 0.5-1.5 kb) were denatured and hybridized at a ratio of 1:100 for the tester to driver DNA. After hybridization had been completed, the products were treated with UDG (which destroyed all the driver DNA) and mung bean nuclease (which digested single stranded DNA and all the non-perfect hybrids). The resulting tester homohybrids were purified, concentrated with streptavidin beads, and subjected to one more round of subtraction. The final PCR product was amplified and cloned in the suitable vector, e.g. pBC KS(+) vector (Stratagene).

From our previous experiments we knew that the NIJ-003 and NL1-401 clones were deleted in this cell line. We isolated DNA from 10 random clones and sequenced them (to perform Southern blotting with these small inserts was impossible due to high the CG content). In this experiment scheme, only short DNA sequences (300-400 bp) were obtained, but their size can be increased using long distance PCR. Two of these clones contained NLJ-003 NotI site.

This experiment demonstrated that subtraction using NotI surrounding sequences is very efficient, since only 2 sites out of 10.000 NotI sites were located in the homozygously deleted region and one of them was found after analysis of only 10 clones. Other clones can be either polymorphic or/and hemizygously deleted since when CODE procedure was applied to the same pair of driver/tester the majority of informative clones (11 of 19) fell under this category.

Thus, the present invention demonstrates that NotI—CODE procedure can be used for enzymes cutting in CpG islands.

Use of NR for NotI Clone Microarrays

Thereafter we decided to check if NR after labelling with ³²P could be directly used for detection of deleted NotI sites. Therefore, we prepared nylon filters with immobilized DNA from NotI linking clones. These filters were hybridized to NR of ACC-LC5 (NR-A) and normal lymphocyte DNA (NR-B).

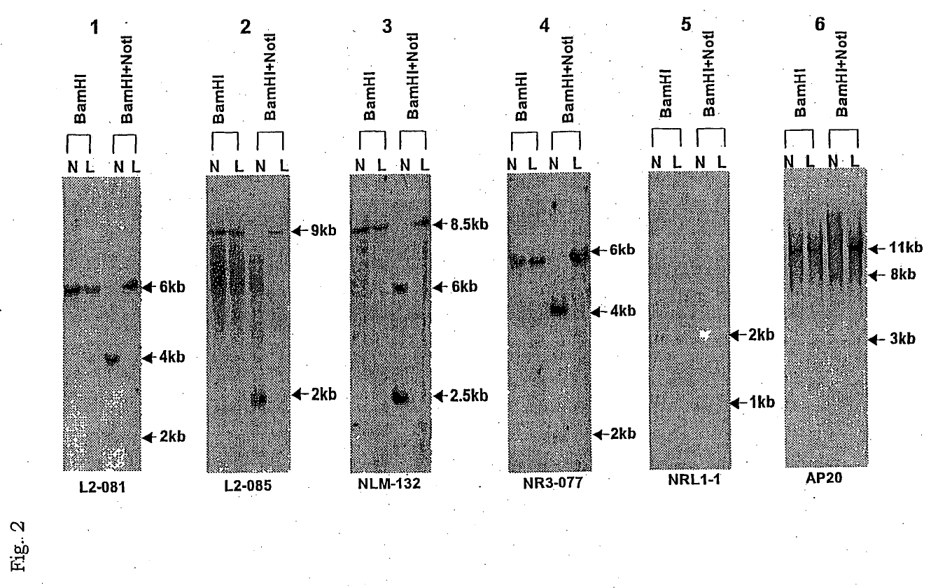

The results showed that these two NRs revealed different hybridization patterns: several clones hybridizing to NR-B did not hybridize to NR-A. First of all it is clear that homozygously deleted NLJ-003 and NL1-401 were easily detected. To understand the reason why other clones failed to hybridize to NR-A, we selected 4 such clones and analysed them using Southern hybridization. Genomic DNA from ACC-LC5 and normal lymphocytes were digested either with BamHI+BglII or with BamHI+BglII+NotI, resolved by electrophoresis in agarose gel, transferred to nylon filter and hybridized to the ³²P labelled insert of a NotI linking clone (FIG. 2:1-4). This experiment demonstrated that all these 4 clones exhibited clear presence of a NotI recognition site in DNA from normal lymphocytes and absence of the corresponding NotI site in ACC-LC5 DNA.

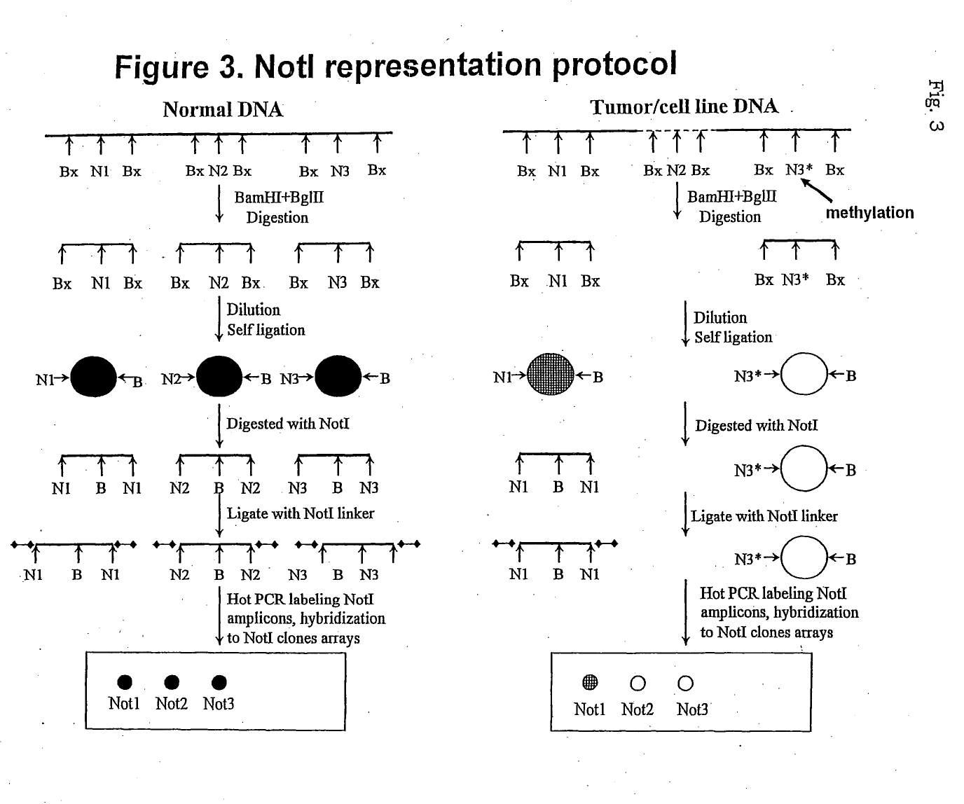

As a next step we performed a similar experiment but used microarrays of DNA from NotI linking clones immobilized to the glass slide. The main idea of this application is shown in FIG. 3. If a particular NotI site is present in the DNA then the circle will be opened with NotI and labelled. However, if this NotI site is deleted or methylated then NR will not contain the corresponding DNA sequences.

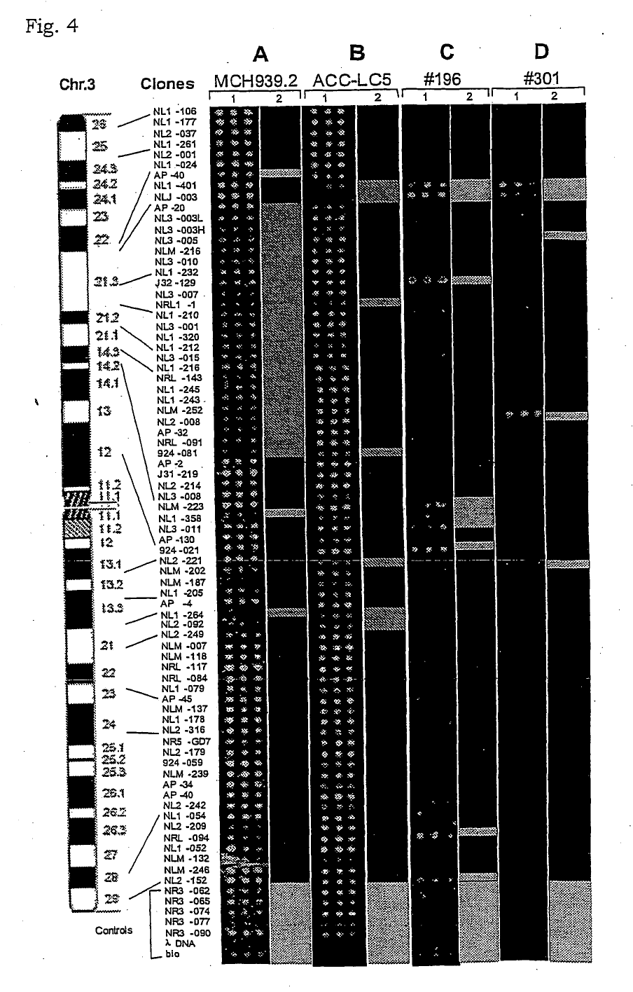

In a first experiment we used DNA isolated from a human-mouse microcell hybrid cell line MCH903.1 (containing the whole human chromosome 3) and MCH939.2 (chr. 3 del p14-p22). NR for MCH903.1 was labelled red and NR for MCH939.3 was labelled green. Thus sequences deleted in MCH939.2 should be red. Thereafter the deletion was precisely mapped (FIG. 4A). Before the present invention, one year of work would have been needed to obtain the same results.

In a second experiment DNA from ACC-LC5 was used again to prepare NR-A and normal lymphocyte DNA was used for making NR-B. NR-A was labelled with Cy3 (green) and NR-B with Cy5 (red). If both sequences are present in both NR then combined colour will be close to yellow and if some clones are deleted in ACC-LC5 then colour for these clones will be more red (FIG. 4B). As it is shown in FIG. 4, homozygously deleted clones NLJ-003 and NL1-401 can unambiguously be detected. Other clones showing redder colour most likely reflect the fact that in practically 100% of the cases SCLC deletion of 3p is detected. Some clones showed the same disbalance as NLJ-003 and NL1-401. This can be explained by methylation of both alleles or deletion of one allele of a NotI site and methylation (or polymorphism) of the other. Indeed, as shown in FIG. 2:3-4, clones NLM-132 and NR3-077 do not contain cleavable NotI sites. In two other cases (AP20 and NRL1-1) that were also completely red, the situation is different. One allele is methylated and the other is deleted (FIG. 2:5-6 and Table 5).

To further check the results of this hybridization. TaqMan probes were designed for 5 NotI linking clones. Quantitative real-time PCR was performed with these primers/probes using ABI Prism□Model 7700 Sequence detector. The results of the quantitative PCR corresponded well with the NotI microarray hybridization, see Table 5 below.

Contamination of tumor DNA with normal DNA represents a serious problem for the identification of tumor suppressor genes, Two RCC biopsies containing 30-40% contaminating normal cells were used in a control experiment to check the sensitivity of NotI microarrays to contamination. One step of the NotI-CODE procedure was used before hybridization, and the probe was labeled with only one dye. As shown in FIG. 4 (C, D), the hybridization clearly identified the two regions most frequently deleted in RCC, 3p21 telomeric (near NLJ-003) and 3p21 centromeric (near NRL1-1). Therefore, the impurity problem that can occur with tumor biopsies can be easily resolved with NotI microarrays.

### EXAMPLES

Cell Lines and General Methods

In the present invention DNA isolated from a small cell lung carcinoma cell line ACC-LC5 was used. This cell line contains homozygous 685-kb deletion in 3p21.3-p22 and was used as a source for DNA A, driver. DNA isolated from normal human lymphocytes was a control DNA (DNA B, tester).

Isolation of DNA, Southern transfer, hybridization, etc. were according to standard methods described in the literature. Construction of Not linking libraries was made as described above.

A standard protocol was used to prepare nylon filter replicas of the gridded NotI linking clones. Nylon filters contained 100 mapped chromosomes specific NotI linking clones and 15 random unmapped human NotI linking clones. For hybridization to nylon filter replicas of the gridded NotI clones, NR probes were ³²-P labeled by PCR.

Sequencing gels were run on ABI 310 automated sequencers (Perkin Elmer) according to the manufacturers' protocols.

Growth of bacteria, other microbiology procedures, isolation of DNA, sequencing was performed according to standard methods.

The Modified NotI—CODE Procedure

Two oligonucleotides: NotX 5′-AAAAGAATGTCAGTGTGTCACGTATGGACGAATTCGC-3′ and NotY: 3′-AAACTTACAGTGTGTGTCACGTATGGCTGCTTAAGCGCCGG-3′ were used to create the NotI linker. Annealing was carried out in a final volume of 100 μl containing 20 μl of 100 μM NotX, 20 μl of 100 μM NotY, 10 μl of 10× M buffer (Boehringer Mannheim) and 50 μl of H₂O. The reaction mixture was boiled for 8 min and allowed to cool slowly at room temperature (r.t.).

Two micrograms of DNA from ACC-LC5 cell line (DNA A) and normal lymphocytes (DNA B) at a DNA concentration of 50 μg/ml were digested with 20 U of BamHI and 20 U of BglII (Boehringer Mannheim) at 37° C. for 5 h, followed by heat-inactivation for 20 min at 65° C. Then 0.4 μg of the digested DNAs were circularized overnight with T4 DNA ligase (Boehringer Mannheim) in the appropriate buffer in 1 ml of the reaction mixture.

DNA was concentrated by precipitation in ethanol, partially filled in with for example Klenow fragment and digested with 10 U of NotI at 37° C. for 3 h. Following digestion, NotI was heat inactivated and DNAs were ligated overnight in the presence of a 50 M excess of NotI linker at room temperature.

PCR of tester amplicon (DNA B with NotI linker) was performed in 100 μl of a solution containing 67 mM Tris-HCl, pH 9.1, 16.6 mM (NH₄)₂SO₄, 1.0 mM MgCl₂, 0.1% Tween 20, 200 μM dNTPs, 100 ng tester amplicon DNA, 400 nM of biotinylated primer NotX and 5U of Taq polymerase.

PCR of the driver amplicon (DNA A with NotI linker) was performed in 20 tubes using the NotX primer and the following modified conditions: dUTP (300 μM) was used instead of dTTP, and 2.5 mM MgCl₂ was used rather than 1.0 mM MgCl₂. The PCR cycling conditions were 72° C. for 5 min, followed by 25 cycles of 95° C. for 1 min, 72° C. for 2.5 min, and a final extension period at 72° C. for 5 min. These PCR amplified tester and driver amplicons we call NotI representation (NR).

All PCR amplified DNA A samples were pooled (2000 μl) and mixed with 20 μl of PCR amplified DNA B (for subtraction we used a ratio of 1:100 of DNA B to DNA A). The pooled sample was concentrated by precipitation in ethanol, purified using a JETquick PCR Purification Spin Kit (GENOMED Inc.), and dissolved in 100 μl H₂O. This DNA mixture was further concentrated to 6 μl and boiled for 10 min under mineral oil.

Subtractive hybridization was performed for 40 h in 9 μl buffer containing 0.4 M NaCl, 100 mM Tris-HCl, pH 8.5 and 1 mM EDTA. After hybridization, the mixture was diluted to 200 μl and extracted with an equal volume of chloroform: isoamyl alcohol (24:1) to remove the mineral oil.

Treatment with UDG (Boehringer Mannheim) was performed in a buffer containing 70 mM Hepes-KOH, pH 7.4, 1 mM EDTA and 1 mM dithiothreitol with 30 U UDG at 37° C. for 4 hrs. Then DNA was precipitated with ethanol and dissolved in 25 μl of TE buffer. To this 3 μl of 10× MBN buffer (30 mM sodium acetate, pH 4.6, 50 mM NaCl, 1 mM zinc acetate and 0.001% Triton X-100) and 20 U of mung bean nuclease (Boehringer Mannheim) were added and incubated at 37° C. for 30 min. The reaction was stopped by the addition of EDTA to a final concentration of 1 mM.

The subtracted DNA was purified with streptavidin coupled Dynabeads M-280 (Dynal A. S, Oslo, Norway) according to the manufacturer's instructions and dissolved in 20 μl of TE buffer. Approximately 0.5 μl of this DNA preparation was PCR amplified as described above for DNA B but using only 8 cycles, before subjecting the amplified DNA to a second round of hybridization.

The final subtraction product was PCR amplified, purified with JETquick PCR Purification Spin Kit (GENOMED Inc.) and digested with NotI. This DNA preparation was inserted into the pBC KS(+) vector (Stratagene), which was digested with NotI and dephosphorylated by alkaline phosphatase (Boehringer Mannheim).

Microarray Preparation, Hybridization and Scanning.

Microarrays were constructed essentially as described by Schena M. et al., 1996. In brief, DNA of NotI linking clones was spotted onto 3-aminopropyl-trimethoxysilane-coated glass microscope slides. Majority of NotI clones contained inserts 2-12 kb (vector part was 3.8 or 4.5 kb, see Zabarovsky et al., 1990). Qiagen-purified DNAs were dissolved in TE and arrayed using GMS 417 Arrayer (Genetic MicroSystems, Woburn, Mass.) with the spot density at 375 μm. The arrays were subsequently air dried, submerged in 70% EtOH for 30 min at room temperature, air dried again, and stored in the dark at −20° C. The microarrays described here contained 150 sequence-validated human chromosome 3-specific STSs in six repetitions, representing 61 known and 49 unknown expressed sequence tags.

The NR probes were labelled in a PCR reaction with the NotX primer. Incorporation of digoxigenin or biotin was done using PCR DIG Labelling Mix (Boehringer Mannheim) or Biotin Reaction Mix (MICROMAX, NEN Life Science Products, Inc., Boston, Mass.). PCR products were purified using MicroSpin PCR Purification Columns (Saveen) and efficiency of the labelling was determined by membrane-based chemiluminescence analysis (MICROMAX, NEN).

Alternative method for preparing NR with low quality DNA was also used. According to this method genomic DNA was simultaneously digested with NotI and another enzyme or combination of enzymes not having CpG pairs in the recognition sites (e.g. Sau3A or BamHI+BglII).

After inactivation of the two enzymes, specific adaptors Sau00N and NBSgt99 were ligated to them:

Sau00N
5′-GATC CTC AAA CGC GT-3′-Amine
3′-GAG TTT GCG CAC AGC ACT GAC CCT TTT GGG ACC-5′
NBSgt99
5′-GGC CTC CAG AAA ACA TCC ACG GGC TCT AGG ATA GAT
CGC-3′
3′-AG GTC TTT TGT AGG-5′

Thereafter, NR was prepared using PCR in the presence of Zuniv and Zgt primers. The PCR cycling conditions were 95° C. for 2 min, followed by 25 cycles of 95° C. for 45 sec, 65° C. for 30 sec and 72° C. for 1.5 min. In general, these NRs showed the same results in hybridization experiments but the background was usually higher.

Qualified Dig- and Bio-labelled probes were combined, denatured at 99° C., 2 min, and hybridized with denatured (0.1M NaOH, 2 min, r.t.) microarrays in the Hybridization Buffer (MICROMAX, NEN) for 5 h at 65° C.

The arrays were washed for 5 min at r.t. in low stringency buffer (0.06×SSC, 0.01% SDS) and developed using TSA system (MICROMAX, NEN) according to the manufacturer's protocols. In brief, we incubated microarrays with anti-DIG antibodies conjugated with horseradish peroxidase (Boehringer Mannheim) and than with Cyanine-3-Tyramide solution. After inactivation of the peroxidase in this first layer, Streptavidin-HRP Conjugate was applied and biotin residues were visualized by Cyanine-5-Tyramide.

The arrays were scanned using GMS 418 Scanner (Genetic MicroSystems, Woburn, Mass.), analyzed and represented by ImaGene 3.05 software (Biodiscovery). Accurate measurements of Cy3/Cy5 fluorescence ratios were obtained by taking the average of the ratios of all six spotted repetitions.

Quantitative Real-Time PCR with TagMan Probes

Oligonucleotide primers and probes were designed to amplify 5 NotI linking clones: NRL1-1 (3p21.2), NL3-001 (3p21.2-21.32), NL1-205 (3p21.2-21.32), NLj3 (3p21.33), 924-021 (3p12.3). huBA—beta-actin gene was used as reference sequence (endogenous control). Final selection of primer and probe sequences, except huBA, was performed using the ABI Primer Express Software Version 1.5 (PE-Applied Biosystems, Foster City, Calif., USA) according to the manufacturer's instruction. TaqMan probes and primers were obtained from Perkin-Elmer. TaqMan probe consists of an oligonucleotide with a 5′-fluorescent reporter dye and a 3′-quencher dye. NLj3, NRL1-1 and hu□A probes contained FAM (6-carboxy-fluoroscein), NL3-001, NL1-205 and 924-021R probes contained JOE (2,7-dimethoxy-4,5-dichloro-6-carboxy-fluoroscein) as reporter dyes, located at the 5′-ends. All reporters were quenched by TAMRA (6-carboxy-N,N,N′,N′-tetramethyl-rhodamine), conjugated to the 3′-terminal nucleotides. The resulting sequences are given below in Table 6

PCR reactions were carried out in 25 μl volumes consisting of 1×PCR buffer A: 10 mM Tris-HCl, 10 mM EDTA, 50 mM KCl, 60 nM passive reference A, pH 8.3 at room temperature; 3.5 mM MgCl₂, 200 μM DATP, dGTP, dCTP, 400 μM dUTP, 100 nM TaqMan probe, forward and reverse primers in appropriate concentrations, 0.025 unit/μl AmpliTaq Gold DNA polymerase, 0.01 unit/μl AmpErase and 5 μl of appropriate diluted DNA template. H₂O was added to 25 μl of total volume. PCR were performed using ABI Prism® Model 7700 Sequence Detector. The reactions were done in triplicate for each sample in the same or separate tubes.

The primer limitation experiments were performed for multiplex PCR with more than one primer pair in the same tube (ABI PRISM 7700 Sequence Detection System. User Bulletin no.2. Relative quantitation of Gene Expression. PE Applied Biosystems, 1997). Thermal cycling conditions consisted of 2 min at 50° C., 10 min at 95° C., followed by 40 cycles of 15 s at 95° C. and 1 min at 60° C.

Cycle threshold (C_{T}) determinations (i.e. calculations of the number of cycles required for reporter dye fluorescence resulting from the synthesis of PCR products to become significantly higher than background fluorescence levels) were automatically performed by the instrument for each reaction.

Details concerning the theory and derivation of the comparative C_{T} method (ΔΔC_{T} method) for target sequence quantitative assessment has been published (ABI PRISM 7700 Sequence Detection System. User Bulletin no.2. Relative quantitation of Gene Expression. PE Applied Biosystems, 1997). This method is dependent upon the inverse exponential relationship that exists between starting quantity (number) of target sequence copies in the reactions and corresponding CT determinations by the ABI7700 system: the more copies, the less value CT (ABI PRISM 7700 Sequence Detection System. User Bulletin no.2. Relative quantitation of Gene Expression. PE Applied Biosystems, 1997). We used an approach referred to as the comparative cycle threshold (CT) method to determine target sequence quantity of tumour sample—ACC-LC5, (target) relative to those in the sample for comparison—normal DNA, (calibrator) and compared with an endogenous control sequence—beta-actin (reference) in both samples. For amplicons designed and optimized according to PE Applied Biosystems 10 guidelines, efficiency is close to 100%. In this case, the amount of target (copy number), normalized to an endogenous reference and relative calibrator, is given by: N_{ACC-LC}5/N_{calibrator}=2^{-ΔΔCT}. The calculation ΔΔC_{T} involves subtraction of mean reference sequence C_{T} values from mean target sequence C_{T} for ACC-LC5 and CBMI, to obtain values ΔC_{T}_{^{ACC-LC5}}=C_{T}_{^{target}}−C_{T}_{^{actin}} and ΔC_{T}_{^{norm}}=C_{T}_{^{target}}−C_{T}_{^{actin}}. The values ΔC_{T}_{^{norm}} are then subtracted from values ΔC_{T}_{^{ACC-LC5}} to obtain ΔΔC_{T}. The range given for all probes relative to β-actin was determined the expression: 2^{-ΔΔCT} with ΔΔC_{T}+s and ΔΔC_{T}−S, where s=the standard deviation of the ΔΔC_{T} value.

For the ΔΔC_{T} calculation to be valid, the efficiency of the target amplification and efficiency of the reference amplification must be approximately equal. Before using the ΔΔCT method for quantitative assessment a validation experiment was performed (ABI PRISM 7700 Sequence Detection System. User Bulletin no.2. Relative quantitation of Gene Expression. PE Applied Biosystems, 1997). The performed validation experiments demonstrated that efficiencies of these targets and references are approximately equal for chosen dilutions. In this case we can use the ΔΔCT calculations for the relative quantitation of target without using standard curves.

Data analysis was done using Sequence Detection System (SDS) software (PE-Biosystems).

The NotI-Passporting Procedure

Two oligonucleotides, BfocII: 5′-ggatgaaaactgga-3′ and Z98NOT: 3′-gtcgtgactgggaaaaccctggcctacttttgacctccgg-5′ were used to create the NotI linker.

Two micrograms of bacterial DNA at a concentration of 50 μg/ml were digested with 20 U NotI (Roche Molecular Biochemicals) at 37° C. for 2 h and heat-inactivated for 20 min at 85° C. Then, 0.4 μg of the digested DNA was ligated to NotI linker (50 M excess) overnight with T4 DNA ligase (Roche Molecular Biochemicals) in the appropriate buffer in 100-μl reaction mixtures. The DNA was then concentrated by precipitation in ethanol and digested with 10 U BpmI at 37° C. for 3 h.

Following digestion, BpmI was heat-inactivated and the DNA was ligated overnight in the presence of a 50 M excess of the ZNBpm linker at room temperature. Two nucleotides, the Zamine: 5′-ctcaaaccgt-3′ and the Z2_univer: 3′-Nngagtttggcacagcactgacccttttgggacc-5′

were used to create the ZNBpm linker.

The sample was then purified using a JETquick PCR Purification Spin Kit (GENOMED Inc.), and dissolved in 100 μl TE. One microliter of this sample was PCR amplified with Z1 univer (3′-gagtttggcacagcactgacccttttgggacc-5′) and antiuniver (5′-cagcactgacccttttgggacc-3′) primers.

PCR was performed in 40 μl solution containing 67 mM Tris-HCl (pH 9.1), 16.6 mM (NH₄)₂SO₄, 2.0 mM MgCl₂, 0.1% Tween 20, 200 μM dNTPs, 3 μl PCR pool, 400 nM of each primer, and 5 U Taq DNA polymerase. The PCR cycling conditions were 95° C. for 1.5 min, followed by 25 cycles of 95° C. for 1 min, 60° C. for 1 min, with 72° C. for 0.5 min, with a final extension period at 72° C. for 3 min.

The final product was purified with the JETquick PCR Purification Spin Kit (Genomed GmbH) and cloned using TOPO TA Cloning kit (Invitrogen AB, Sweden). Sequencing gels were run on ABI 377 automated sequencers (Perkin Elmer), according to the manufacturers' protocols, using standard primers.

For the analysis of the complex flora composition, we suggest using only some specific fragments of the genomes (e.g. NotI representations, NotI tags, NotI linking clones, etc.). Thus we do not aim to sequence all genomes or study all genes. We append special signatures for the particular microorganism/genes and analyze these signatures in different samples of colon flora. In the present invention study work we have analyzed the use of short sequence tags appended to NotI or other restriction enzyme recognition site. The collection of NotI tags represents NotI sequence passport or in short NotI passport and NotI passporting means creation of NotI tags/passports. The naming is based on the initially used enzymes, but the methods can be adapted to other restriction enzymes as well.

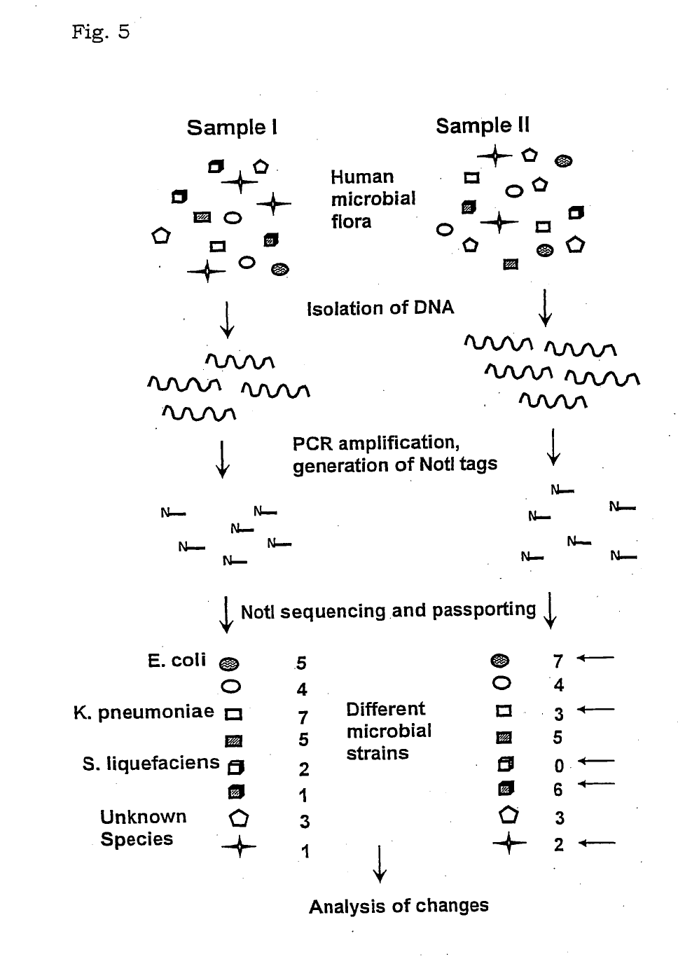

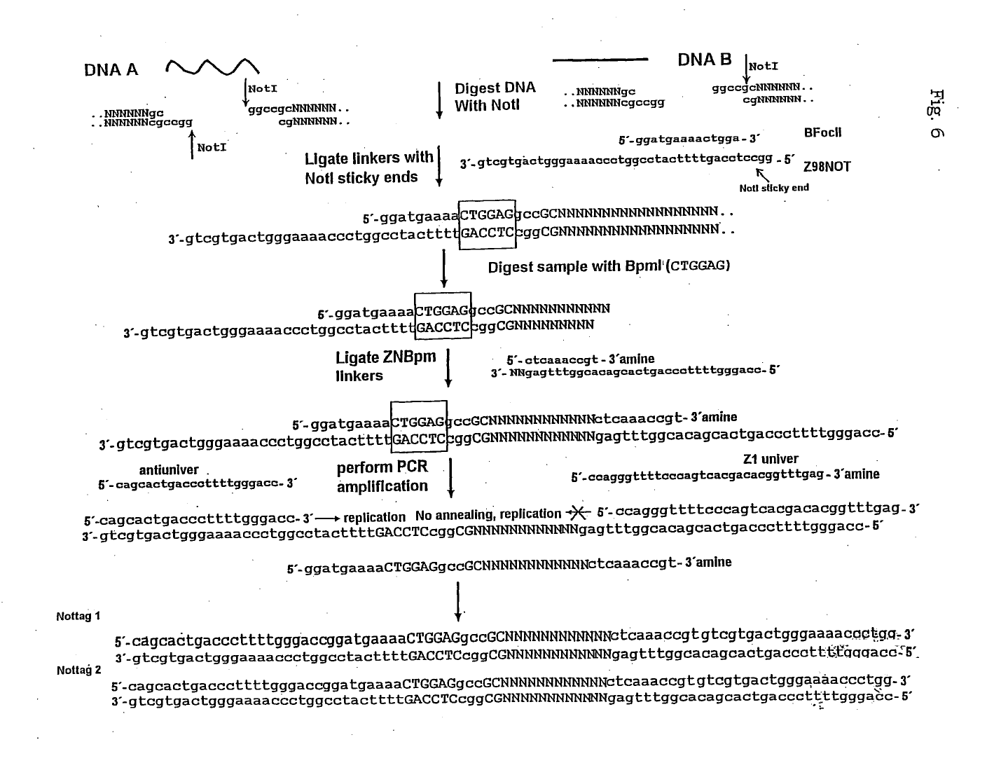

The general design of the experiment is as follows (FIG. 5). DNA generated from faecal samples and surgical specimens are digested with NotI and ligated to special linker containing BpmI recognition site. Then DNA is digested with BpmI, ligated to the special linkers and PCR amplified. We have proved that in these conditions only specific 85 bp NotI-BpmI fragments are amplified (FIG. 6). After digestion with BpmI and FokI this fragment will generate 24 bp fragments which represent particular NotI sites. From here it is possible to work in two directions.

a) Concatemer Strategy

The 24 bp units will be ligated into the concatemers of about 1.000 bp size, cloned and sequenced. Each sequencing reaction will give information about 20-50 NotI sites.

b) Oligomer Strategy.

New high-throughput sequencing techniques, such as pyrosequencing or massively parallel signature sequencing have been developed recently. They allow one person to produce many thousands sequences per day. However, these sequences are very short 20-40 bp and suit our needs well, whereby NotI passport for the particular specimen can be produced. Comparing these passports from e.g. different individuals or from the same individual before and after drug treatment we find the difference between them. This information in some cases can be directly used to make conclusions. In other cases, using these sequences we can identify NotI linking clones which are different between two samples. These clones can be used for further analysis, e.g. finding the genes which are responsible for a certain medical condition (e.g. cancer, aging etc.) or sequencing/isolation of the required microorganism.

TABLE 1
Comparison of different microarrays to study genome copy number changes and methylation.*

| | | CGH | | | | RST |
|---|---|---|---|---|---|---|
| Method/ | | (BACs, P1, | | | | (NotI |
| Feature | cDNA | PACs) | Representation | SNP | CGI | microarrays) |
| Homozygous | Low | Yes | Yes/NO | NO | Yes/NO | Yes |
| deletions | | | | | | |
| Hemizygous | Low | Yes | NO | NO | NO | Yes |
| deletions | | | | | | |
| LOH | NO | NO | Yes | Yes | Yes/NO | Yes |
| Ampli- | Low/ | Yes | Yes | NO | Yes | Yes |
| fication | Medium | | | | | |
| Methylation | NO | NO | NO | NO | NO | Yes |
| Number of | More than | 10.000-30.000 | 1.500 | 1.300 | 1.500 | 10.000-20.000 |
| available | 40.000 | | (polymorphic | (can be increased) | (polymorphic BglII | |
| markers | | | BglII fragments | | fragments per | |
| | | | per genome) | | genome) | |
| Connection | Direct | Indirect | NO (indirect) | NO (indirect) | NO (indirect) | Direct |
| to genes | | | | | | |
| Main | Low sensitivity | Very | Not convenient | High sensitivity to | Not convenient for | High CG content; |
| disad- | and precision; | expensive, | for large-scale | normal cell | large-scale | small size of the |
| vantages | small size of | difficulties | screening; small | contamination, | screening; small | inserts; |
| | the insert; | to work with | size of the | short hybridizing | size of the inserts; | discrimination |
| | high | large-insert | inserts; only 1% | sequences; many | only 1% | between LOH, |
| | background, | vectors: | polymorphic | reactions and | polymorphic sites; | deletions/amplifications |
| | several EST | low yield, | sites; unknown | primers are needed; | unknown location; | and |
| | markers | rearrangements; | location; 2.5% | expensive; less than | 2.5% of all DNA is | methylation |
| | should be | 100% DNA | of all DNA is | 30% of markers are | labeled, unknown | |
| | used to | is labeled | labeled, | polymorphic | purification from | |
| | determine | (many | unknown | | repeats. | |
| | copy number; | repeats) | purification | | | |
| | 100% DNA is | | from repeats. | | | |
| | labeled (many | | | | | |
| | repeats) | | | | | |
| Main | Direct | Good to | Can be easily | Very small fraction | Can be easily | Methylation |
| advantages | connection to | check copy | adopted for | of the genome is | adopted for small | detection; up to |
| | RNA profiling | number | small scale | labeled; good to | scale experiments | 45% of clones are |
| | | changes | experiments | check LOH | (small genomes) | polymorphic, easy |
| | | | (small genomes) | | | to solve normal |
| | | | | | | cell contamination, |
| | | | | | | one reaction and |
| | | | | | | one pair of primers |
| | | | | | | are used; |
| | | | | | | comparatively |
| | | | | | | cheap, good to |
| | | | | | | check LOH and |
| | | | | | | copy number |
| | | | | | | changes, only 0.1-0.2% |
| | | | | | | of the |
| | | | | | | genome is labeled; |
| | | | | | | 10 fold purification |
| | | | | | | from repeats; |
| | | | | | | direct connection |
| | | | | | | to genes; |
| | | | | | | simultaneous |
| | | | | | | detection of |
| | | | | | | different |
| | | | | | | aberrations |
| | | | | | | associated with |
| | | | | | | cancer |
| | | | | | | development |

*Efficiency of the method to detect the particular feature

TABLE 2
Comparison of NotI and CGI microarrays

| Feature | NotI microarrays | CGI-microarrays |
|---|---|---|
| Uncomplete | No effect | Artificial result |
| restriction | | |
| digestion | | |
| Specificity of | 0.1-0.5% of the total | 100% total human DNA |
| labeling | human DNA | |
| Repeats | 10% compared to the | Approximately the same as |
| | average in human | in average |
| | genome | |
| rRNA genes | No | Yes |
| Homozygous | Yes | No |
| deletions | | |
| Hemizygous | Yes | No |
| deletions | | |
| Hemizygous | Yes | No |
| methylation | | |
| Oligo microarrays | Yes | ??? |
| Homozygous | Yes | Yes |
| methylation in | | |
| cancer cells | | |
| Quality of clones | All sequenced, all | Partly sequenced, many |
| | contain genes | repeated sequences and |
| | | repeats like LINE etc. |
| Number of | >5.000 | Unknown |
| available clones | | |

TABLE 3
The number of recognition sites for rare cutting restriction enzymes in selected bacterial genomes

| | GENOME | SIZE | NotI | PacI | PmeI | SbfI | SgfI | SgrAI | Sse2321 | SwaI |
|---|---|---|---|---|---|---|---|---|---|---|
| 1 | Bacillus subtilis | 4, 2 | 81 | 89 | 89 | 51 | 51 | 157 | 52 | 176 |
| 2 | Borrelia burgdorferi | 1, 5 | 1 | 234 | 37 | 8 | 0 | 2 | 0 | 548 |
| 3 | Campylobacter jejuni | 1, 6 | 0 | 91 | 42 | 13 | 5 | 1 | 0 | 526 |
| 4 | Chlamydophila pneumoniae AR39 | 1, 2 | 2 | 59 | 10 | 21 | 13 | 4 | 1 | 60 |
| 5 | Deinococcus radiodurans R1 | 3, 3 | 15 | 1 | 4 | 28 | 7 | 645 | 164 | 1 |
| 6 | Escherichia coli K12 | 4, 6 | 23 | 143 | 87 | 68 | 222 | 548 | 31 | 117 |
| 7 | Escherichia coli O157:H7 | 5, 5 | 36 | 165 | 92 | 108 | 239 | 642 | 34 | 126 |
| 8 | Helicobacter pylori 26695 | 1, 7 | 7 | 32 | 35 | 4 | 88 | 61 | 12 | 67 |
| 9 | Helicobacter pylori J99 | 1, 6 | 14 | 34 | 43 | 4 | 87 | 66 | 15 | 76 |
| 10 | Lactococcus lactis subsp. lactis | 2, 4 | 3 | 176 | 47 | 17 | 2 | 11 | 0 | 235 |
| 11 | Rickettsia prowazekii | 1, 1 | 1 | 239 | 20 | 10 | 1 | 4 | 0 | 229 |
| 12 | Staphylococcus aureus Mu50 | 2, 9 | 0 | 440 | 83 | 12 | 5 | 12 | 2 | 602 |
| 13 | Streptococcus pneumoniae R6 | 2, 0 | 1 | 40 | 25 | 30 | 1 | 9 | 0 | 51 |
| 14 | Synechocystis PCC6803 | 3, 6 | 44 | 192 | 104 | 40 | 3160 | 182 | 18 | 167 |
| 15 | Vibrio cholerae | 4, 0 | 73 | 103 | 117 | 37 | 203 | 199 | 24 | 104 |

TABLE 4
Specificity of restriction tags in E. coli and H. pylori strains.

| | | Cutting sites in | | | | Unique for | | | | Unique for | | | |
|---|---|---|---|---|---|---|---|---|---|---|---|---|---|
| | | the genome | | | | the species | | | | the strain | | | |
| Species | Strain | PmeI | SbfI | NotI | Total | PmeI | SbfI | NotI | Total | PmeI | SbfI | NotI | Total |
| Escherichia | K12 (4.6 Mb) | 87 | 68 | 23 | 178 | 74 | 61 | 20 | 155 | 25 | 26 | 6 | 57 |
| coli | | | | | | | | | | | | | |
| | O157H7 (5.5 Mb) | 92 | 108 | 36 | 236 | 77 | 90 | 34 | 201 | 28 | 55 | 20 | 103 |
| Helicobacter | 26695 (1.7 Mb) | 35 | 4 | 7 | 46 | 35 | 4 | 7 | 46 | 21 | 2 | 4 | 27 |
| pylori | | | | | | | | | | | | | |
| | J99 (1.6 Mb) | 43 | 4 | 14 | 61 | 43 | 4 | 14 | 61 | 33 | 2 | 11 | 46 |

TABLE 5
Relative quantitative measurements using comparative
(ΔΔC_{T}) method for normal lymphocyte DNA
and ACC-LC5 cell line

| | | N_{ACC-LC5}/ | |
|---|---|---|---|
| Target/colour | Location | Nₙₒᵣₘ = 2^{-ΔΔCT} | Comments |
| 924-021/yellow | 3p12.3 | 0.94 (0.83-1.05) | No changes |
| NRL1-1/red | 3p21.2 | 0.51 (0.41-0.62) | Initial target |
| | | | sequence copy |
| | | | number in |
| | | | ACC-LC5 is |
| | | | half of what is |
| | | | obtained in CBMI |
| | | | (hemizygous |
| | | | deletion) |
| NL3-001/yellow | 3p21.2-21.32 | 1.12 (0.98-1.26) | No changes |
| NL1-205/yellow | 3p21.2-21.32 | 1.25 (0.75-1.74) | No changes |
| NLj3/red | 3p21.33 | 0.00 | Zero sequence |
| | | | copy number |
| | | | (homozygous |
| | | | deletion) |

TABLE 6
TaqMan probe, primer sequences and product lengths

| | | | |
|---|---|---|---|
| | | | Amplicon, |
| Target | Oligonucleotide | Sequence (5′ → 3′) | bp |

| | | | |
|---|---|---|---|
| 924-021 | 924-021, probe | TGCTGGCCACAGGCCCTGC | 52 |
| (3p12.3) | primer(F) | TGCATGTGCCAGTGTTGATAAA | |
| | primer(R) | GTGTTGTGAGCCCTGGGAA | |
| NRL1-1 | NRL1-1, probe | AGCCTGAGCTGGGCAGACAGTTTCC | 74 |
| (3p21.2) | primer(F) | CAGCCCCACGGTCACTTC | |
| | primer(R) | GCCAAAACAGACCCAGCCT | |
| NL3-001 | NL3-001, probe | CCCCAGAAACGCGCGGGC | 60 |
| (3p21.2 -21.32) | primer(F) | CTTGCCATCTGCAATTCCCT | |
| | primer(R) | CTCCATGAGGCTGTGGGAAG | |
| NL1-205 | NL1-205, probe | GCGGCTGGCTCTGCGC | 63 |
| (3p21.2 -21.32) | primer(F) | ATGAGGCTCTTTCCCATGCC | |
| | primer(R) | GCCGGATTCAGGATGCTTT | |
| NLj3 | NLj3, probe | CTGGCGGAGAGACTGGGAGCGA | 125 |
| (3p21.33) | primer(F) | CAGAGTGCGTGTGCCGACT | |
| | primer(R) | ACAACTTCTCTGCGGGCGT | |
| hu?A | hu?A, probe | ATGCCCCCCCCATGCCATCCTGCGT | 295 |
| (control) | primer(F) | TCACCCACACTGTGCCCATCTACGA | |
| 7 chromosome | primer(R) | CAGCGGAACCGCTCATTGCCAATGG | |

### REFERENCES

Bicknell, D. C., Markie, D., Spurr, N. K., Bodmer, W. F., 1991. The human chromosome content in human x rodent somatic cell hybrids analyzed by a screening technique using Alu PCR. Genomics 10, 186-192.
Brookes, A. J., 1999. The essence of SNPs. Gene 234, 177-186.
Costello J F, Fruhwald M C, Smiraglia D J, Rush L J, Robertson G P, Gao X, Wright F A, Feramisco J D, Peltomaki P, Lang J C, Schuller D E, Yu L, Bloomfield C D, Caligiuri M A, Yates A, Nishikawa R, Su Huang H, Petrelli N J, Zhang X, O'Dorisio M S, Held W A, Cavenee W K, Plass C. Aberrant CpG-island methylation has non-random and tumour-type-specific patterns. Nat Genet 2000 24(2): 132-138
Espinosa-Urgel, M., Kolter, R., 1998. Escherichia coli genes expressed preferentially in an aquatic environment. Mol. Microbiol. 28, 325-332.
Ishikawa, S., Kai, M., Tamari, M., Takei, Y., Takeuchi, K., Bandou, H., Yamane, Y., Ogawa, M., Nakamura, Y., 1997. Sequence analysis of a 685-kb genomic region on chromosome 3p22-p21.3 that is homozygously deleted in a lung carcinoma cell line. DNA Res. 4, 35-43.
Kaiser, C., Von Stein, O., Laux, G., Hoffmann M., 1999. Functional genomics in cancer research: identification of target genes of the Epstein-Barr virus nuclear antigen 2 by subtractive cDNA cloning and high-throughput differential screening using high-density agarose gels. Electrophoresis 20, 261-268.
Li, J., Wang, F., Zabarovska, V., Wahlestedt, C., Zabarovsky, E. R., 1999. Cloning of polymorphisms (COP): enrichment of polymorphic sequences from complex genomes. Nucleic Acids Res., in press.
Lisitsyn, N., Lisitsyn, N., Wigler, M., 1993. Cloning the differences between two complex genomes. Science 259, 946-951.
Lisitsyn, N. A., Segre, J. A., Kusumi, K., Lisitsyn, N. M., Nadeau, J. H., Frankel, W. N., Wigler, M. H., Lander, E. S., 1994. Direct isolation of polymorphic markers linked to a trait by genetically directed representational difference analysis. Nat. Genet. 6: 57-63.
Lisitsyn, N. A., Lisitsina, N. M., Dalbagni, G., Barker, P., Sanchez, C. A., Gnarra, J., Linehan, W. M., Reid, B. J., Wigler, M. H., 1995. Comparative genomic analysis of tumours: detection of DNA losses and amplification. Proc. Natl. Acad. Sci. USA 92: 151-155.
Parikh, V. S., Morgan, M. M., Scott, R., Clements, L. S., Butow, R. A., 1987. The mitochondrial genotype can influence nuclear gene expression in yeast. Science 235: 576-580.
Rosenberg, M., Przybylska, M., Straus, D., 1994. ‘RFLP subtraction’: a method for making libraries of polymorphic markers. Proc. Natl. Acad. Sci. USA, 91: 6113-6117.
Sambrook, J., Fritsch, E. F., Maniatis, T., 1989. Molecular Cloning: A Laboratory Manual, Cold Spring Harbor Laboratory Press, Cold Spring Harbor, N.Y.
Sugai, M., Kondo, S., Shimizu, A., Honjo, T., 1998. Isolation of differentially expressed genes upon immunoglobulin class switching by a subtractive hybridization method using uracil DNA glycosylase. Nucleic Acids Res. 26, 911-918.
Sugimura T, Ushijima T. Genetic and epigenetic alterations in carcinogenesis. Mutat Res (2000) 462(2-3): 235-246.
Yamakawa, K., Takahashi, T., Horio, Y., Murata, Y., Takahashi, E., Hibi, K., Yokoyama, S., Ueda, R., Takahashi, T., Nakamura, Y., 1993. Frequent homozygous deletions in lung cancer cell lines detected by a DNA marker located at 3p21.3-p22. Oncogene 8, 327-330.
Yan P S, Chen C M, Shi H, Rahmatpanah F, Wei S H, Caldwell C W, Huang T H. Dissecting complex epigenetic alterations in breast cancer using CpG island Microarrays. Cancer Res 2001 61(23): 8375-8380
Zabarovsky, E. R., Boldog, F., Thompson, T., Scanlon, D., Winberg, G., Marcsek, Z., Erlandsson, R., Stanbridge, E. J., Klein, G., Sumegi, J., 1990. Construction of a human chromosome 3 specific NotI linking library using a novel cloning procedure. Nucleic Acids Res. 18, 6319-6324.
Zabarovska, V., Li, J., Muravenko, O., Fedorova, L., Ernberg, I., Wahlestedt, C., Klein, G. and Zabarovsky, E. R. CIS—cloning of identical sequences between two complex genomes. Chromosome Research, in press.
Allikmets et al.: NotI linking clones as tools to join physical and genetic mapping of the human genome. Genomics, (1994) 19: 303-309.
Kashuba et al.: Analysis of NotI linking clones isolated from human chromosome 3 specific libraries. Gene, 1999, 239: 259-271.
Katouli et al. Composition and diversity of intestinal coliform flora influence bacterial translocation in rats after hemorrhagic stress. Infection and immunity 62: 4768-4774, 1994
Klein, J. (1999). Batrachomyomachia: frog 1, mice 0. Scand J Immunol, 49: 11-13.
Li J. et al.: COP—a new procedure for cloning single nucleotide polymorphisms. Nucleic Acids Res. (2000) 28, e1,p. i-v.
Li et al.: CODE: a new genomic subtraction method for cloning deleted sequences. Biotechniques, (2001), 31: 788-793.
Midtvedt, T. (1999). In L. A. Hansson and R. H. Yolken (eds.), Microbial functional Activities. Lippincott-Raven, Philadelphia, Vol. Nestle Nutritional Workshop Series, pp. 79-96.
Ronaghi et al. Pyrosequencing—a DNA sequencing method based on real-time pyrophosphate detection. Science (1998) 281: 363-365.
Sandberg et al.: Capturing whole-genome characteristics in short sequences using a naïve Bayesian classifier. Genome Res. 2001, 11:1404-1409.
Velculesku et al.: Serial analysis of gene expression. Science (1995) 270:484-487.
Zabarovska et al.: Slalom libraries: a new approach to genome mapping and sequencing. Nucleic Acids Res. (2002) 30 (e6): 1-8.
Zabarovsky et al.: Construction of a human chromosome 3 specific NotI linking library using a novel cloning procedure. Nucl. Acids Res., 1990, 18:6319.
Zabarovsky et al.: A new strategy for mapping the human genome based on a novel procedure for constructing jumping libraries. Genomics, 1991, 11: 1030-1039.
Zabarovsky et al.: NotI clones in the analysis of human genome. Nucl. Acids Res., (2000) 28: 1635-1639.
Zabarovsky et al.: Novel techniques to identify the species composition of complex microbial systems: restriction site tagged microarrays (RST) and NotI signatures. Microecology and Therapy, in press.

1. Method for preparing nucleic acid or and/or modified nucleic acid reference material bound to a solid phase, comprising steps of:
digesting nucleic acid and/or modified nucleic acid reference material using biochemical and/or chemical approaches, to obtain sequence fragments surrounding a specific restriction enzyme recognition site,
selecting said nucleic acid and/or modified nucleic acid sequence fragments flanking a specific restriction enzyme recognition site.

2. Method according to claim 1, wherein said reference material is digested by a first restriction enzyme and/or one or more second restriction enzymes.

3. Method according to claim 2, wherein the restriction enzymes are endonucleases.

4. Method according to claim 3, wherein the recognition sites of the first endonuclease is scarcely distributed along said genomic material and is located adjacent to gene sequences, and the recognition sites of said one or more second restriction endonucleases are more frequently occurring along said genomic material than the sites of the first endonuclease.

5. Method of claim 4, wherein the digestion by the first and second restriction endonucleases are performed simultaneously, and different linkers are ligated to the ends resulting from cutting by the first and second restriction endonucleases, respectively, which linkers are designed such that when primers are added in order to make PCR reactions, only the fragments containing ends resulting from cutting by the first restriction endonuclease will be amplified.

6. Method of claim 4, wherein the reference material is first digested by the one or more second restriction endonucleases, the ends of the thus obtained fragments are self-ligated into the form of circular nucleic acid and/or modified nucleic acid molecules, and any linear fragments remaining after self-ligation are inactivated before digestion with the first restriction endonuclease, whereby the linear fragments resulting from the digestion by the first endonuclease are subjected to PCR amplification.

7. Method of claim 2, wherein the first restriction endonuclease in NotI, or any other restriction endonuclease, the restriction sites of which occurs in proximity to CpG islands in the genomic material.

8. Method of claim 2, wherein the first restriction endonuclease is NotI, PmeI or Sbfl, or a combination of two or more of said endonucleases, and the second endonuclease is BamHI, BclI, BglII or Sau3A, or a combination of two or more of said endonucleases.

9. Method according to claim 1, wherein said nucleic acid and/or modified nucleic acid reference material is selected from RNA, DAN, peptides or modified oligonucleotides, or a combination of two or more of said materials.

10. Method according to claim 1, wherein the solid phase is a glass slide, coded beads, cellulose, such as nitrocellulose, or filters.

11. Method of claim 1, wherein the genomic material is derived from one or more humans, from different locations in the body/bodies and at the same or different points in time.

12. Method of claim 1, wherein the genomic material is derived from bacteria from the gut, skin or other parts of the human body.

13. Method of claim 1, wherein the genomic material is derived from any organism, bacteria, animal, or plant, or product produced there from, or from any substance wherein genomic material can be contained, especially air and water.

14. Use of representation of the genome, or of a part thereof, of an organism, comprising multiple copies of the nucleic acid and/or modified nucleic acid fragments, or a selection thereof, obtained by means of the method of claim 1 in discriminating between different genomes, detecting methylations, deletions, mutations and other changes within genomic material obtained from the same individual at different points of time, or in the genomic material obtained from one individual as compared to a standard representation obtained from at least one other individual, or a combination thereof.

15. Use of the representation according to claim 14, wherein the representation in liquid form is hybridized to the nucleic acid and/or modified nucleic acid fragments present in the form of said solid phases.

16. Use of the representation of the genome, or of a part thereof, of an organism, comprising multiple copies of the nucleic acid and/or modified nucleic acid fragments, or a selection thereof, obtained by means of the method of claim 1 for:
studying methylation and copy number changes in eukaryotic genomes for diagnosis, prognosis, identification of cancer causing genes, etc,
genotyping different microorganisms (viruses, prokaryotic, eukaryotic),
studying biocomplexity and diversity of complex biological systems, i.e. human gut, bacterial flora in water, food, air resources,
identifying pathogenic organisms in different sources including complex biological mixtures,
producing passports (images of microarrays hybridizations, database containing tag sequences) for different purposes: to describe organisms at different conditions, i.e. different ages, disease/healthy, infected/uninfected etc,
identifying new organisms, e.g. bacterial species,
producing microarrays (DNA-and oligo-based) to study all above described features,
verification and maintenance of large biological collections/banks, i.e. verifying cell lines and individual organisms for higher organisms and confirming the purity of the particular strain for microbial species,
producing kits for labeling and hybridization with microarrays,
producing kits for making sequence tagging (passporting), and
producing oligo microarrays to analyze sequence tags.

17. Use of the representation according to claim 16, wherein the representation in liquid form is hybridized to the nucleic acid and/or modified nucleic acid fragments present in the form of said solid phases.

18. NotI genomic subtraction method for cloning deleted sequences (CODE-genomic subtraction method) based on the use of fragments obtained by the method for preparing nucleic acid or and/or modified nucleic acid reference material bound to a solid phase, comprising the steps of
digesting nucleic acid and/or modified nucleic acid reference material using biochemical and/or chemical approaches, to obtain sequence fragments surrounding a specific restriction enzyme recognition site,
selecting said nucleic acid and/or modified nucleic acid sequence fragments flanking a specific restriction enzyme recognition site.

19-20. (canceled)
